(19) United States
(12) Patent Application Publication
(10) Pub. No.: US 2004/0151678 A1
(43) Pub. Date: Aug. 5, 2004

(54) Hair care compositions

(76) Inventors: Jean-Pierre Barrere, Le Chesnay (FR); Florence Vaudeleau, Boulogne (FR)

Correspondence Address: YOUNG & THOMPSON, 745 SOUTH 23RD STREET 2ND FLOOR, ARLINGTON, VA, 22202

(21) Appl. No.: 10/257,115

(22) PCT Filed: Mar. 30, 2001
(86) PCT No.: PCT/EP01/03643

(30) Foreign Application Priority Data
Apr. 10, 2000 (FR) 0004570

(51) Int. Cl. A61K 7/06; A61K 7/13

(52) U.S. Cl. 424/70.1; 8/406

(57) ABSTRACT

The present invention relates to hair care cosmetic compositions comprising Zanthoxylum bungeanum extracts as active ingredient together with cosmetically acceptable excipients, optionally containing hair perming or hair dyeing agents. The Zanthoxylum extracts are obtained by extraction of Zanthoxylum bungeanum pericarp and further fractionation using CO₂ in super-critical conditions.

The present invention relates to cosmetic compositions comprising Zanthoxylum extracts.

More specifically, the present invention relates to cosmetic formulations useful in hair care formulations, containing Zanthoxylum extracts as the sole vegetable ingredient.

Zanthoxylum bungeanum Maxim. (Rutaceae) is a perennial plant native to China. It is widely used in the country of origin as a spice (Sichuan peppercorn, huajiao) for the treatment of gastrointestinal pains; in folk medicine the essential oil of this plant is reported as strong insect repellant.

Recently, EP 568001 disclosed the antiviral activity of the skin of this fruit.

In U.S. Pat. No. 5,137,912 chelerythrine, extracted from the root bark of Zanthoxylum simulans (synonym of bungeanum), was disclosed to be active in the prevention of thrombosis. According to JP 01294657, the extraction of the pericarp with organic solvents provides an extract containing isobutylamides exerting local anaesthetic effect already 30 seconds after the application on the tongue and lasting up to 20-80 minutes.

The extracts used in the present invention, prepared by extraction and further fractionation using CO₂ in supercritical conditions, as disclosed in WO 00/02570, are devoid of the local anaesthetic activity characteristic of the extracts obtained by means of solvents.

### TECHNICAL BACKGROUND

It is well known by hair technicians and by their costumers that a major problem with permanent waving treatments (in the following “perming treatments”) of hair as well as with either permanent or non-permanent dyeing of hair (in the following referred to as “hair treatments”) is the irritation that conventional hair perming or hair dyeing agents (in the following referred to as “chemicals”) induce on the scalp.

Such irritation may vary from very mild discomfort to itching sensation to stinging, burning, redness, depending on the chemicals used for such “hair treatments” as well as on the inherent sensitivity of the scalp of the single individual.

Many attempts have been made to reduce the irritation and itching caused by the chemicals used in “hair treatments”, but they proved to be rather unsatisfactory.

It would therefore be highly desirable to provide compounds able to prevent or reduce the sensations of discomfort, irritation and itching caused by the above mentioned “hair treatments”.

It has now surprisingly been found that extracts from Zanthoxylum bungeanum exert such an effect on the scalp.

The present invention, therefore, relates to the use of Zanthoxylum extracts as active ingredient exerting a soothing effect to prevent or reduce the sensations of discomfort, irritation and itching of the scalp caused by “hair treatments”, as well as to cosmetic formulations containing them.

Preferred embodiments of the invention make use of Zanthoxylum extracts obtained by extraction of Zanthoxylum bungeanum pericarp and further fractionation using CO₂ in supercritical conditions, as disclosed in WO 00/02570.

Said extracts can be used in the form of solutions in long chain fatty alcohols, preferably oleyl alcohol, in concentrations ranging from 5 to 25% by weight. Said solutions can in their turn be formulated into emulsions, non-alcoholic solutions, fluid solutions, and the like.

The compositions of the invention comprise Zanthoxylum extracts together with a suitable vehicle for the topical administration. More precisely, the compositions of the invention may be formulated in the solid, semi-solid or liquid forms. Solid and semi-solid formulations comprise, for example, creams, foams, gels, and the like. Liquid formulations can be, for example, in the form of aqueous or oily solutions or suspensions, oil-in-water emulsions, lotions, shampoos, milks, rinses etc.

The Zanthoxylum extracts will be present in the compositions of the invention preferably in amounts of 1.0 to 1% by weight.

The compositions of the invention will further contain conventional excipients, for example cosmetic oils, surfactants, detergents, emulsifying agents, emollients, antioxidants, thickeners, pH control agents, UV absorbers, conditioners, perfumes, dyes, preservatives, and the like.

According to a further embodiment, the compositions of the invention will also contain conventional hair perming or hair dyeing agents.

The present invention also relates to a cosmetic method of preventing, reducing or eliminating the sensation of discomfort, irritation and itching of the scalp, comprising the topical application of a composition containing Zanthoxylum extracts in a soothing effective amount.

The compositions of the invention will be applied to the scalp either prior to the treatment with the chemical, or simultaneously with such chemical, to prevent, reduce or soothe its irritant effect. In the first case, the composition of the invention will be preferably applied to the scalp 15 to 30 minutes prior to the “hair treatment”. In the second case, the compositions of the invention will either be applied simultaneously with the chemical or they will contain the chemical itself.

The characteristics of the compositions of the invention will be further illustrated in the following experimental section.

### EXPERIMENTAL SECTION

Evaluation of the Soothing Effect of Zanthalene^{(R)} (Zanthoxylum Alatum in Olyel Alcohol) in Subjects Receiving Salon Hair Coloration and Perming Treatments

The study was carried out on 20 volunteers (women): all of them had their hair subjected to colouring and perming treatments.

Before beginning the study on the volunteers, the following parameters were evaluated:

pH of the composition of the invention (0.5% Zanthalene^{(R)} formulation);

analysis of its formula to detect any possible interactions with the dye and perming products;

compatibility of the dye and perming products by a test on a lock of hair.

Furthermore, the conditions of the scalp were evaluated by the hairdresser, in terms of: dandruff, irritation, itching, stinging, redness, etc.

For each volunteer:

one half of the head was treated with Zanthalene^{(R)};

one half of the head was left untreated or was treated with a placebo lotion.

The placebo had the following composition:

Blank hair lotion solution (placebo)

| INCI Name | Trade Name | |
|---|---|---|
| Oleyl alcohol | HD Eutanol V (Henkel) | 0.50% |
| Polysorbate 20 | Tween 20 (ICI surfact.) | 2.00% |
| Alcohol | Alcohol | 25.00% |
| Disodium EDTA | Disodium EDTA | 0.10% |
| Aqua | Water | q.s. to 100% |

Hair lotion with Zanthalene^{(R)} 20% solution

| INCI Name | Trade Name | |
|---|---|---|
| Zanthoxylum Bungeanum, | Zanthalene(R), | 0.50% |
| oleyl alcohol | 20% solution | |
| Polysorbate 20 | Tween 20 (ICI surfact.) | 2.00% |
| Alcohol | Alcohol | 25.00% |
| Disodium EDTA | Disodium EDTA | 0.10% |
| Aqua | Water | q.s. to 100% |

Application of both products on each studied zone was carried out 20 to 30 minutes prior hair treatment. Afterwards, hair dyeing treatment was carried out on both-parts of the head by the hairdresser responsible for the study.

Subsequently, volunteers answered the subjective questionnaire regarding their sensations. The hairdresser evaluated the aspect of the scalp of the volunteers after colouring: dandruff, irritation, itching sensation.

Statistical Method

Results were tested with the null hypothesis: the two lotions have the same probability to be chosen (p=½), so that the results follow the binomial distribution. Volunteers who indicated that the treatments are equal were included in the negative group.

The risk α was fixed at 0.05.

20 women out of 20 felt a discomfort sensation with the “BLANK SOLUTION”. With the “0.5% Zanthalene solution”, 18 women (90%) felt no discomfort, while the remaining 20% felt a mild discomfort.

The difference between the two lotions on discomfort was statistically significant.

The soothing effect was evaluated in terms of:

a) time of appearance

b) intensity

c) preference for one of the two products used.

a) The soothing effect appeared earlier for:

16 volunteers with the lotion “0.5% Zanthalene solution”,

1 volunteer with the lotion “BLANK SOLUTION”.

The soothing effect appeared at the same time for 3 volunteers.

The soothing effect had an earlier onset of action in a statistically significant way with the lotion “0.5% Zanthalene solution” than with the lotion “BLANK SOLUTION”.

b) Intensity of the soothing effect:

17 volunteers indicated a more intense soothing effect when treated with the lotion “0.5% Zanthalene solution”,

2 volunteers indicated that the soothing effect was the same for both lotions, and

one volunteer had no opinion on this question.

The soothing effect of the lotion “0.5% Zanthalene solution” was statistically significant more intense than the soothing effect with the lotion “BLANK SOLUTION”.

c) Preferred product

The lotion “0.5% Zanthalene solution” was preferred by 17 volunteers and the lotion “BLANK SOLUTION” by one volunteer. 2 Volunteers had no preference.

The lotion “0.5% Zanthalene solution” was preferred in a statistically significant way in comparison to the lotion “BLANK SOLUTION” by the volunteers.

The hairdresser indicated that both lotions did not affect the appearance of the hair of the volunteers (glossy, volume, colour . . . ).

Similar results were obtained also in the case of perming treatments without dying treatments and in the case of dying treatments without perming treatments.

1. Hair care cosmetic compositions comprising, as the sole vegetable active ingredient, Zanthoxylum bungeanum extracts as active ingredient, together with cosmetically acceptable excipients.

2. Compositions as claimed in claim 1, wherein the Zanthoxylum bungeanum extracts are obtained by extraction of Zanthoxylum bungeanum pericarp and further fractionation using CO₂ in supercritical conditions.

3. Cosmetic compositions as claimed in claims 1 and 2, wherein said extracts are in the form of solutions in long chain fatty alcohols in concentrations ranging from 5 to 25% by weight.

4. Cosmetic compositions as claimed in claim 3, wherein the long chain fatty alcohol is oleyl alcohol.

5. Cosmetic compositions as claimed in any one of the above claims, wherein the Zanthoxylum extracts are present in concentrations ranging from 0.11 to 10% by weight.

6. Cosmetic compositions as claimed in any one of the above claims, in the solid, semi-solid or liquid forms.

7. Cosmetic compositions as claimed in claim 6, wherein said compositions are selected from the group consisting of cream, foam, gel, aqueous or oily solution or suspension, oil-in-water emulsion, lotion, shampoo, conditioner, milk, rinse.

8. The use of a cosmetic composition as claimed in any one of the above claims, prior to perming treatments of hair or either perm anent or non-permanent dyeing, of hair.

9. Cosmetic compositions as claimed in any one of claims 1 to 7, further containing conventional hair perming, or hair dyeing agents.

10. A method of cosmetic treatment for providing a soothing effect to prevent or reduce the sensations of discomfort, irritation and itching of the scalp caused by perming treatments of hair as well as by either permanent or non-permanent dyeing of hair, which method comprises administering topically to the scalp of a subject a cosmetic composition as claimed in claims 1-9.
